(19) United States
(12) Patent Application Publication
(10) Pub. No.: US 2025/0012763 A1
(43) Pub. Date: Jan. 9, 2025

(54) A SYSTEM AND METHOD OF NON-CONTACT MEASUREMENT OF ONE OR MORE MECHANICAL PROPERTIES OF A MATERIAL

(71) Applicant: Agency for Science, Technology and Research, Singapore (SG)

(72) Inventors: Lei ZHANG, Singapore (SG); Lin KE, Singapore (SG)

(21) Appl. No.: 18/704,834

(22) PCT Filed: Oct. 26, 2022
(86) PCT No.: PCT/SG2022/050770

§ 371 Date: Apr. 25, 2024

(30) Foreign Application Priority Data
Oct. 28, 2021 (SG) 10202112031V

(51) Int. Cl. G01N 29/24; A61B 8/10; G01N 29/04; G01N 29/07; G01N 33/483

(52) U.S. Cl. CPC G01N 29/2418; G01N 29/041; G01N 29/07; G01N 33/4833; A61B 8/10; G01N 2291/02827

(57) ABSTRACT

There is provided a system and a method of non-contact measurement of one or more mechanical properties of a material, the system comprising an ultrasonic module comprising an ultrasonic applicator configured to apply ultrasonic pressure on a target region of the material; a detection module comprising an electromagnetic wave emitter and an electromagnetic wave detector, said electromagnetic wave emitter being configured to emit an incident beam of electromagnetic waves towards the target region of the material, and said electromagnetic wave detector being configured to detect an emergent beam of electromagnetic waves reflected from the target region and/or transmitted through the target region; and a processing module configured to determine one or more measures corresponding to the one or more mechanical properties of the material, based on changes in the emergent beam of electromagnetic waves.

### TECHNICAL FIELD

The present disclosure relates broadly to a system and method of non-contact measurement of one or more mechanical properties of a material.

### BACKGROUND

In general, the measurement of mechanical properties of a material faces various kinds of challenges. Such challenges may include requirements for non-destructive measurement of the material (e.g., cases where the sample material is expensive or rare, cases where the same sample material is to be re-used after measurement, or cases where the sample material is a part of a living subject and must not be damaged or destroyed by the measurement). Such challenges may also include requirements for measuring changes in mechanical properties of the material as a function of parameters such as time, temperature, hydration level etc. Such challenges may further include requirements for simplicity of use, safety, and the ability to provide fast, precise and accurate measurements.

In particular, the measurement of mechanical properties of viscoelastic materials such as hydrogel has generated considerable interest. Hydrogels are made of three-dimensional hydrophilic polymer network, which can intake and hold large amount of water contents. Due to its similarity to biological tissues, it is widely used in many industries and biological processes. One of the important applications of hydrogels is to use as constructs for engineering tissues. Mechanical properties such as the Young's modulus and shear modulus of the hydrogels are generally recognized as important parameters for tissue growth. Poor mechanical strength of hydrogels usually leads to tissues with unacceptable mechanical properties.

In addition, as hydration levels are time-dependent, it is a challenge to monitor changes in the mechanical properties of hydrogels. The current available methods for measurement of hydrogel mechanical properties include extensiometry, compression, bulge, and indention tests. These methods may be supplemented with optical measurement and finite element simulations to calculate the mechanical properties of the hydrogels. The current available methods are suitable for small coupon tests in an in-vitro manner. However, these methods are not suitable for in-vivo tests where contact may not be possible. In addition, the current available non-contact methods at best provide indirect parameters and do not provide direct measures of mechanical properties.

The human eye can be viewed as natural hydrogels. The measurement of ocular elastic properties is more challenging because of the requirements to perform the measurement in a non-invasive, in-vivo and ideally non-contact manner. The ocular rigidity (OR) is affected not only by the outer ocular coats (i.e., sclera and cornea) but also by the choroid and the status of ocular blood circulation. In addition, OR may be altered by surgical procedures affecting the ocular walls, such as refractive procedures. According to several previous studies, OR may, in turn, affect the accuracy of measurements of the intraocular pressure (IOP) as well as the pathogenesis of various ophthalmic conditions, such as glaucoma or age-related macular degeneration (AMD). Although a variety of methods, such as intraoperative anterior chamber manometry, axial length-associated OR measurement, ultrasound elastography, measurement of pulse amplitude and fundus pulse, have been employed for the quantitative determination of OR, technical difficulties in obtaining accurate in vivo measurements have, until now, limited its clinical role. There are currently no satisfactory in-vivo cornea elastic/rigidity and structure change measurement methods, and there is an unmet need to extract mechanical properties of hydrogels with the hydration level changes.

Thus, there is a need for a system and method of non-contact measurement of one or more mechanical properties of a material, which seek to address or at least ameliorate one of the above problems.

### SUMMARY

In one aspect, there is provided a system for non-contact measurement of one or more mechanical properties of a material, the system comprising an ultrasonic module comprising an ultrasonic applicator configured to apply ultrasonic pressure on a target region of the material; a detection module comprising an electromagnetic wave emitter and an electromagnetic wave detector, said electromagnetic wave emitter being configured to emit an incident beam of electromagnetic waves towards the target region of the material, and said electromagnetic wave detector being configured to detect an emergent beam of electromagnetic waves reflected from the target region and/or transmitted through the target region; and a processing module configured to determine one or more measures corresponding to the one or more mechanical properties of the material, based on changes in the emergent beam of electromagnetic waves.

In one embodiment, the electromagnetic wave emitter and the electromagnetic wave detector are positioned such that the electromagnetic wave detector is capable of detecting the emergent beam of electromagnetic waves reflected from the target region.

In one embodiment, the electromagnetic wave emitter and the electromagnetic wave detector are positioned such that the electromagnetic wave detector is capable of detecting the emergent beam of electromagnetic waves transmitted through the target region.

In one embodiment, the electromagnetic wave emitter is positioned to emit an incident beam of electromagnetic waves that is substantially perpendicular to the surface of the material at the target region.

In one embodiment, the ultrasonic applicator comprises an air-coupled ultrasonic transducer.

In one embodiment, the ultrasonic applicator comprises an array of transducer elements configured to generate directed ultrasonic waves to a 3D spatial location at the target region.

In one embodiment, the ultrasonic applicator comprises a pulsed laser device configured to emit pulses of electromagnetic waves for applying the ultrasonic pressure at the target region on the surface of the material.

In one embodiment, the electromagnetic wave emitter is configured to emit an incident beam of electromagnetic waves having a frequency falling in a range from 0.1 THz to 10 THz.

In one embodiment, the processing module is configured to perform time-domain spectrum measurement based on changes in the emergent beam of electromagnetic waves detected by the electromagnetic wave detector.

In one embodiment, the one or more measures corresponding to the one or more mechanical properties of the material comprises a measure of elasticity, rigidity, viscoelasticity and/or rheology.

In one aspect, there is provided a method of non-contact measurement of one or more mechanical properties of a material, the method comprising, emitting an incident beam of electromagnetic waves from an electromagnetic wave emitter towards a target region of the material; applying or varying an ultrasonic pressure on the target region of the material; detecting an emergent beam of electromagnetic waves reflected from the target region and/or transmitted through the target region with an electromagnetic wave detector; and determining one or more measures corresponding to the one or more mechanical properties with a processing module, based on changes in the emergent beam of electromagnetic waves.

In one embodiment, the changes in the emergent beam of electromagnetic waves are determined with respect to a reference emergent beam detected at a different time point.

In one embodiment, the ultrasonic pressure applied at the target region on the surface of the material is substantially constant without amplitude modulations.

In one embodiment, the method comprises, (i) emitting the incident beam of electromagnetic waves and detecting the emergent beam of electromagnetic waves at a first time point; (ii) varying the ultrasonic pressure on the target region of the material; (iii) emitting the incident beam of electromagnetic waves and detecting the emergent beam of electromagnetic waves at a second time point; (iv) repeating steps (i) to (iii) one or more times; and (v) determining the one or more measures corresponding to the one or more mechanical properties based on differences in emergent beams at at least two different time points.

In one embodiment, the ultrasonic pressure is applied using an air-coupled ultrasonic transducer over air.

In one embodiment, the ultrasonic pressure is applied using a pulsed laser device over air, by applying pulses of electromagnetic waves having a pulse duration in the order of nanoseconds, picoseconds, or femtoseconds, and optionally wherein the pulses of electromagnetic waves are diffused to cover the target region on the surface of the material.

In one embodiment, the incident beam of electromagnetic waves emitted by the electromagnetic wave emitter has a frequency falling in a range from 0.1 THz to 10 THz.

In one embodiment, the method further comprises performing time-domain spectrum measurement based on the emergent beam of electromagnetic waves detected by the electromagnetic wave detector before and after application of the ultrasonic pressure.

In one embodiment, the material comprises a hydrogel or a soft tissue in an eye of a mammalian subject.

In one aspect, there is provided a computer readable storage medium having stored thereon instructions for instructing a processing unit of a system to execute a method of non-contact measurement of one or more mechanical properties of a material, the method comprising, emitting an incident beam of electromagnetic waves from an electromagnetic wave emitter towards a target region of the material; applying or varying an ultrasonic pressure on the target region of the material; detecting an emergent beam of electromagnetic waves reflected from the target region and/or transmitted through the target region with an electromagnetic wave detector; and determining one or more measures corresponding to the one or more mechanical properties with a processing module, based on changes in the emergent beam of electromagnetic waves.

### Definitions

The term “ultrasonic” or “ultrasound” as used herein refers to a frequency in the range from about 15 kHz to about 100 MHz. In one example, an upper limit for air-coupled ultrasound waves may be about 1 MHz. In another example, an upper limit for laser-generated ultrasound waves may be about 100 MHz.

The term “hydrogel” as used herein is to be interpreted broadly to refer to water-swollen networks of polymers.

The term “ex vivo” or “in vitro” as used herein is to be interpreted broadly to refer to an environment outside a living organism, such as a human or other animal.

The term “in vivo” as used herein is to be interpreted broadly to refer to an environment within a living organism, such as a human or other animal.

The term “micro” as used herein is to be interpreted broadly to include dimensions from about 1 micron to about 1000 microns.

The term “nanometer” as used herein is to be interpreted broadly to include dimensions less than about 1000 nm.

The term “nanosecond” as used herein is to be interpreted broadly to refer to a unit of time equal to 10-9 of a second. For example, a nanosecond laser refers to a laser that emits ultrashort pulses of light with a pulse duration on the order of nanoseconds.

The term “picosecond” as used herein is to be interpreted broadly to refer to a unit of time equal to 10-12 of a second. For example, a picosecond laser refers to a laser that emits ultrashort pulses of light with a pulse duration on the order of picoseconds.

The term “femtosecond” as used herein is to be interpreted broadly to refer to a unit of time equal to 10-15 of a second. For example, a femtosecond laser refers to a laser that emits ultrashort pulses of light with a pulse duration on the order of femtoseconds.

The term “terahertz” as used herein is to be interpreted broadly to refer to an electromagnetic wave having a frequency of from about 0.1 THz to about 10 THz.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated. Coupling may be used to describe, for example, of two or more objects, devices, and/or components that are communicatively coupled, mechanically coupled, and/or electrically coupled. The term “communicatively coupled” generally refers to any type or configuration of coupling that places two or more objects, devices, components, or portions, elements, or combinations thereof in communication. Mechanical and electrical communications are examples of such communications. The term “mechanically coupled” generally refers to any physical binding, adherence, attachment, and/or other form of physical contact between two or more objects, devices, components, or portions, elements, or combinations thereof. The term “electrically coupled” indicates that one or more objects, devices, components, or portions, elements, or combinations thereof, are in electrical contact such that an electrical signal, pulse, or current (e.g., electrical energy) is capable of passing between the one or more objects, enabling the objects to electrically communicate with one another.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

The description herein may be, in certain portions, explicitly or implicitly described as algorithms and/or functional operations that operate on data within a computer memory or an electronic circuit. These algorithmic descriptions and/or functional operations are usually used by those skilled in the information/data processing arts for efficient description. An algorithm is generally relating to a self-consistent sequence of steps leading to a desired result. The algorithmic steps can include physical manipulations of physical quantities, such as electrical, magnetic or optical signals capable of being stored, transmitted, transferred, combined, compared, and otherwise manipulated.

Further, unless specifically stated otherwise, and would ordinarily be apparent from the following, a person skilled in the art will appreciate that throughout the present specification, discussions utilizing terms such as “scanning”, “calculating”, “determining”, “replacing”, “generating”, “initializing”, “outputting”, and the like, refer to action and processes of an instructing processor/computer system, or similar electronic circuit/device/component, that manipulates/processes and transforms data represented as physical quantities within the described system into other data similarly represented as physical quantities within the system or other information storage, transmission or display devices etc.

The description also discloses relevant device/apparatus for performing the steps of the described methods. Such apparatus may be specifically constructed for the purposes of the methods, or may comprise a general purpose computer/processor or other device selectively activated or reconfigured by a computer program stored in a storage member. The algorithms and displays described herein are not inherently related to any particular computer or other apparatus. It is understood that general purpose devices/machines may be used in accordance with the teachings herein. Alternatively, the construction of a specialized device/apparatus to perform the method steps may be desired.

In addition, it is submitted that the description also implicitly covers a computer program, in that it would be clear that the steps of the methods described herein may be put into effect by computer code. It will be appreciated that a large variety of programming languages and coding can be used to implement the teachings of the description herein. Moreover, the computer program if applicable is not limited to any particular control flow and can use different control flows without departing from the scope of the invention.

Furthermore, one or more of the steps of the computer program if applicable may be performed in parallel and/or sequentially. Such a computer program if applicable may be stored on any computer readable medium. The computer readable medium may include storage devices such as magnetic or optical disks, memory chips, or other storage devices suitable for interfacing with a suitable reader/general purpose computer. In such instances, the computer readable storage medium is non-transitory. Such storage medium also covers all computer-readable media e.g., medium that stores data only for short periods of time and/or only in the presence of power, such as register memory, processor cache and Random Access Memory (RAM) and the like. The computer readable medium may even include a wired medium such as exemplified in the Internet system, or wireless medium such as exemplified in Bluetooth technology. The computer program when loaded and executed on a suitable reader effectively results in an apparatus that can implement the steps of the described methods.

The example embodiments may also be implemented as hardware modules. A module is a functional hardware unit designed for use with other components or modules. For example, a module may be implemented using digital or discrete electronic components, or it can form a portion of an entire electronic circuit such as an Application Specific Integrated Circuit (ASIC). A person skilled in the art will understand that the example embodiments can also be implemented as a combination of hardware and software modules.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a system and method of non-contact measurement of one or more mechanical properties of a material are disclosed hereinafter.

### System for Non-Contact Measurement of One or More Mechanical Properties of a Material

In various embodiments, there is provided a system for non-contact measurement of one or more mechanical properties of a material, the system comprising an ultrasonic module comprising an ultrasonic applicator configured to apply/exert ultrasonic pressure on a target region of the material; a detection module comprising an electromagnetic wave emitter and an electromagnetic wave detector, said electromagnetic wave emitter being configured to emit an incident beam of electromagnetic waves towards the target region of the material, and said electromagnetic wave detector being configured to detect an emergent beam of electromagnetic waves reflected from the target region and/or transmitted through the target region; and a processing module configured to determine one or more measures corresponding to the one or more mechanical properties of the material, based on changes in the emergent beam of electromagnetic waves. In various embodiments, the detection module, ultrasonic module and processing module are electrically coupled to one another.

In various embodiments, the target region may be on a surface of the material. In various embodiments, the material may be a solid or a semi-solid, e.g., gel-like solid. In various embodiments, the material is one that has a high water content, e.g., no less than about 20 wt % of water, no less than about 30 wt % of water, no less than about 40 wt % of water, no less than about 50 wt % of water, no less than about 60 wt % of water, no less than about 70 wt % of water, no less than about 80 wt % of water, or no less than about 90 wt % of water. In general, water has a broadband absorption of electromagnetic radiation in the frequency range from about 2 THz to about 3 THz. There are also resonant line absorption spectra of water spread across the THz range of frequencies. Accordingly, in various embodiments, the system or associated method disclosed herein is capable of performing mechanical analysis of materials, e.g., hydrogels, with high water concentrations, e.g., hydrogels having a water content from about 70% to about 98%.

In various embodiments, the material may be a viscoelastic material. In various embodiments, the material may be a hydrogel, e.g., hydrogel mixture. In various embodiments, the material may be a biological tissue. In various embodiments, the biological tissue may be from a mammalian subject, including but not limited to rats, mice, cats, dogs, horses, sheep, cattle, cows, pigs, rabbits, non-human primates, and humans. In various embodiments, the biological tissue may be a soft biological tissue. Examples of soft biological tissue include but are not limited to eye tissue, e.g., cornea, sclera, and choroid tissues. In various embodiments, the biological tissue may be a hard biological tissue. Examples of hard biological tissue include but are not limited to bone, tooth, dentin, and cementum. In various embodiments, non-contact measurement of one or more mechanical properties of the biological tissue may be performed in-situ/in-vivo and ex-vivo/in vitro. In various embodiments, measurements performed in-situ/in-vivo refer to measurements done in or on tissue from an organism in its natural environment and conditions. In various embodiments, measurements performed ex-vivo/in vitro refer to measurements done in or on tissue from an organism in an external environment with minimal alteration of natural conditions.

In various embodiments, the mechanical properties of the material include but are not limited to elasticity, rigidity, viscoelasticity, and rheology properties. In various embodiments, the one or more measures of mechanical properties include but are not limited to a modulus of elasticity (i.e., Young's modulus and shear modulus), a modulus of rigidity (i.e., stiffness), viscoelastic properties (i.e., dynamic storage and loss moduli) and rheology properties (e.g., relaxation times and compliance). In various embodiments, the mechanical properties of the material (e.g., elasticity and rigidity) may change as a result of changes in hydration level of the material. In various embodiments, the changes in hydration level of the material may occur over time. In various embodiments, the changes in hydration level of the material may result in a corresponding change in the emergent beam of electromagnetic waves. In other words, in various embodiments, changes in the emergent beam of electromagnetic waves may correlate with changes in hydration levels of the material. In various embodiments therefore, the system or associated method disclosed herein may be configured to measure/evaluate the hydration level of a material. In various embodiments therefore, the system may advantageously be used to monitor changes in the one or more mechanical properties of the material due to changes in the hydration level of the material. In various embodiments therefore, the system or associated method disclosed herein may advantageously be used to monitor changes in the one or more mechanical properties of the material at different time points.

In various embodiments, the system and method utilize a combination of time-domain spectroscopy (e.g., terahertz time-domain spectrum measurement) and ultrasonic pressure loading to measure one or more mechanical properties of a material. In various embodiments, the system and method may advantageously provide a rapid and non-contact technique for measuring one or more mechanical properties of a material. In various embodiments, the system and method may even more advantageously provide a non-invasive and non-destructive technique for measuring one or more mechanical properties of a material, thereby rendering the system and method suitable for in-situ/in-vivo measurements. In various embodiments, the system is a portable system. By portable, it is meant, among other things, that the system is capable of being transported relatively easily. The system may have an overall size and/or weight which allows it to be transported relatively easily. In various embodiments, the system and method may be employed in applications such as measurement of mechanical properties of a material, measurement of biosamples with high water content, evaluation of hydration level of a material, tissue hardness test, monitoring and measurement of tissue e.g., cornea properties.

In various embodiments, the detection module is configured to perform time-domain spectroscopy, e.g., terahertz (THz) time-domain spectroscopy. In various embodiments, the electromagnetic wave emitter (i.e., illumination system) is configured to emit electromagnetic waves and the electromagnetic wave detector (i.e., detection system) is configured to detect electromagnetic waves in the terahertz (THz) range of the electromagnetic spectrum which lies between the microwave and infrared frequencies. In various embodiments, the electromagnetic wave emitter may be configured to emit an incident beam (i.e., illumination beam) of electromagnetic waves having a frequency falling in a range from about 0.1 THz (10¹¹ Hz, 3 mm wavelength) to about 10 THz (10¹³ Hz, 3.3 μm wavelength). In various embodiments, the electromagnetic wave detector may be configured to detect an emergent beam (i.e., return beam) of electromagnetic waves having a frequency falling in a range from about 0.1 THz (10¹¹ Hz, 3 mm wavelength) to about 10 THz (10¹³ Hz, 3.3 μm wavelength). In various embodiments, the incident beam and the emergent beam of electromagnetic waves may have frequencies falling in a range selected from the following group of numbers: 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, and 10 THz. In various embodiments, the emission and detection scheme of time-domain spectroscopy is based on the sample's effect on both the amplitude and the phase of the terahertz radiation (e.g., change in amplitude and phase of the emergent/detected beam vs that of the incident/emitted beam). In various embodiments, analysis of the one or more mechanical properties is based on both the amplitude change and phase change of the incident beam (i.e., incident THz radiation) and emergent beam (i.e., reflected/transmitted THz radiation). In various embodiments, the effective frequency range of the electromagnetic waves may vary according to different types of material compositions, e.g., hydrogel chemical compositions. In various embodiments, the frequency range with less absorption would be more efficient for mechanical performance evaluation.

In various embodiments, the electromagnetic wave emitter is configured to emit the incident beam of electromagnetic waves over the air towards the target region of the material. In other words, there may be no physical contact between the electromagnetic wave emitter and the material. In various embodiments, the electromagnetic wave detector is configured to detect the emergent beam of electromagnetic waves reflected from the target region of the material and thereafter transmitted over the air, and/or transmitted through the target region of the material and thereafter transmitted over the air. In other words, there may be no physical contact between the electromagnetic wave detector and the material.

In various embodiments, the electromagnetic wave emitter and the electromagnetic wave detector may be positioned such that the electromagnetic wave detector is capable of detecting the emergent beam of electromagnetic waves reflected from the target region. In various embodiments, the ultrasonic applicator, electromagnetic wave emitter, and electromagnetic wave detector may be configured to be positioned on the same side of the material, such that the electromagnetic wave detector detects the emergent beam of electromagnetic waves reflected from the target region of the material. In various embodiments, the emergent beam of electromagnetic waves may be reflected from the surface of the material at the target region.

In various embodiments, the electromagnetic wave emitter and the electromagnetic wave detector may be positioned such that the electromagnetic wave detector is capable of detecting the emergent beam of electromagnetic waves transmitted through the target region. In various embodiments, the ultrasonic applicator and electromagnetic wave emitter may be configured to be positioned on a first side of the material, and the electromagnetic wave detector may be configured to be positioned on a second opposite side of the material, such that the electromagnetic wave detector detects the emergent beam of electromagnetic waves transmitted through the target region. In various embodiments, the emergent beam of electromagnetic waves may be transmitted through the material at the target region (i.e., passes through the entire thickness of the material).

In various embodiments, the electromagnetic wave emitter and the electromagnetic wave detector may be positioned such that the electromagnetic wave detector is capable of detecting the emergent beam of electromagnetic waves reflected from and transmitted through the target region. In various embodiments, there may be more than one electromagnetic wave detector. Accordingly, in some embodiments the emergent beam of electromagnetic waves reflected from and transmitted through the target region may be detected. In various embodiments, the ultrasonic applicator, electromagnetic wave emitter, and a first electromagnetic wave detector may be configured to be positioned on a first side of the material, such that the electromagnetic wave detector detects the emergent beam of electromagnetic waves reflected from the target region of the material; and a second electromagnetic wave detector may be configured to be positioned on a second opposite side of the material, such that the electromagnetic wave detector detects the emergent beam of electromagnetic waves transmitted through the target region.

In various embodiments, the electromagnetic wave emitter may be positioned to emit an incident beam of electromagnetic waves at an incident angle to the material at the target region. In various embodiments, the incident angle is defined as the angle between the incident beam and the normal to the surface of the material at the target region. The incident angle may be from about 0° to about 60°. The incident angle may fall in a range with start and end points selected from the following group of numbers: 0, 5, 10, 15, 20, 25, 30, 35, 40, 45, 50, 55, and 60°. In various embodiments, the electromagnetic wave emitter may be positioned to emit an incident beam of electromagnetic waves that is substantially perpendicular to the surface of the material at the target region (i.e., the incident angle is 0°).

In various embodiments, the electromagnetic wave detector may be positioned to detect an emergent beam of electromagnetic waves at an emergent angle to the material at the target region. In various embodiments, the emergent angle is defined as the angle between the emergent beam and the normal to the surface of the material at the target region. The emergent angle may be from about 0° to about 60°. The emergent angle may fall in a range with start and end points selected from the following group of numbers: 0, 5, 10, 15, 20, 25, 30, 35, 40, 45, 50, 55, and 60°. In various embodiments, the electromagnetic wave detector may be positioned to detect an emergent beam of electromagnetic waves that is substantially perpendicular to the surface of the material at the target region (i.e., the emergent angle is 0°).

In various embodiments, the detection module further comprise a controller/control unit configured to monitor and control optical signals (i.e., incident beams of electromagnetic waves) emitted from the electromagnetic wave emitter, and optical signals (i.e., emergent beams of electromagnetic waves) detected at the electromagnetic wave detector. In various embodiments, the controller may be a THz controller configured to monitor and control THz generation and detection. In various embodiments, the controller of the detection module is coupled to the electromagnetic wave emitter and electromagnetic wave detector. In various embodiments, the controller of the detection module is further coupled to the processing module.

In various embodiments, the system further comprises an optical module for forming an optical path. In various embodiments, the optical module may be configured to relay and direct at least a portion of the incident beam emitted by the electromagnetic wave emitter along a first optical path onto the target region of the material. The optical module may be further configured to relay and direct at least a portion of the emergent beam reflected from and/or transmitted through the target region of the material along a second optical path to be received/detected by the electromagnetic wave detector. The optical module may comprise an assembly of optical elements/devices arranged in the first optical path and the second optical path to relay and direct at least a portion of the incident beam and emergent beam. The optical elements may include but are not limited to lens, filter, reflector, mirror, and beam splitter.

In various embodiments, the ultrasonic module is configured to apply/exert/project an ultrasonic/ultrasound pressure on the material, e.g., target region of the material. In various embodiment, the ultrasonic module is configured to apply the ultrasonic pressure over the air on the material (e.g., air-coupled ultrasound). In various embodiments, there is no physical contact between components of the ultrasonic module and the material.

In various embodiments, the ultrasonic pressure is configured to generate/produce a change in one or more properties of the material, including but not limited to optical property (e.g., refraction index) and mechanical property (e.g., deformation). In various embodiments, the ultrasonic pressure is configured to generate/produce stress at the target region of the material. For example, the ultrasonic pressure applied on the material may produce mechanical deformation of the material, said mechanical deformation resulting in a change in one or more characteristics of the emergent beam detected by the electromagnetic wave detector. For example, the ultrasonic pressure applied on the material may produce a change in the refractive index of the material, said change in refractive index resulting in a change in one or more characteristics of the emergent beam detected by the electromagnetic wave detector. The ultrasonic pressure applied on the materials may also produce a continuous or pulsed vibration at a specific frequency in the material which leads to dynamic changes in the refractive index at the said specific frequency, which in turn result in a change in one or more characteristics of the emergent beam detected by the electromagnetic wave detector. In various embodiments, the generation of ultrasonic pressure and the detection of electromagnetic waves may be synchronized. With synchronization between the ultrasonic generation and electromagnetic wave detection, phase delay between the applied ultrasonic pressure and the electromagnetic wave could be quantified and related to the viscoelastic and rheology properties at the said frequency.

In various embodiments, the ultrasonic module comprises an ultrasonic applicator configured to apply/exert/project ultrasonic pressure on a target region of the material. In various embodiments, the ultrasonic module and/or system does not comprise or is devoid of ultrasound probes or detectors to detect reflected ultrasound waves.

In various embodiments, the ultrasonic module may comprise an ultrasonic applicator in the form of an ultrasound/ultrasonic transducer, e.g., an air-coupled ultrasound/ultrasonic transducer. In various embodiments, the transducer may be coupled to an ultrasonic driver comprising a function generator or signal generator, and an amplifier. In various embodiments, the function generator or signal generator is configured to send an electrical signal to the ultrasonic transducer, which converts the electrical signal to ultrasound energy, i.e., ultrasound waves. In various embodiments, the air-coupled ultrasonic transducer is configured to transmit ultrasound waves over the air (i.e., air-transmitted ultrasound waves) to the target region of the material, thereby exerting an ultrasonic pressure on the material. Thus, in various embodiments, the ultrasonic module/applicator is an over-the-air ultrasound module/application and is capable of exerting ultrasonic pressure over the air or a gas medium to the target region without a need for a separate liquid or a solid intermediate medium. This advantageously allows the ultrasonic pressure to be exerted on the material in a non-contact manner. Even more advantageously, the ultrasonic transducer is safe for use in in-vivo measurements, e.g., in-vivo measurement on the eye.

In various embodiments, the ultrasound waves transmitted by the ultrasonic applicator may have a frequency from about 15 kHz to about 100 KHz. In various embodiments, the ultrasound waves transmitted by the ultrasonic applicator may fall in a range with start and end points selected from the following group of numbers: 15 kHz, 20 kHz, 25 kHz, 30 kHz, 35 kHz, 40 kHz, 45 kHz, 50 kHz, 55 kHz, 60 kHz, 65 kHz, 70 KHz, 75 kHz, 80 kHz, 85 kHz, 90 kHz, 95 kHz, 100 kHz, 200 kHz, 300 kHz, 400 kHz, 500 kHz, 600 kHz, 700 kHz, 800 kHz, 900 kHz, 1 MHZ, 2 MHZ, 3 MHZ, 4 MHZ, 5 MHz, 6 MHZ, 7 MHz, 8 MHz, 9 MHZ, 10 MHz, 20 MHZ, 30 MHz, 40 MHz, 50 MHz, 60 MHz, 70 MHz, 80 MHZ, 90 MHZ, and 100 MHz. In one example, an upper limit for air-coupled ultrasound waves may be about 1 MHz. In another example, an upper limit for laser-generated ultrasound waves may be about 100 MHz.

In various embodiments, the ultrasonic transducer may be configured to generate ultrasound waves having an intensity that is proportional to an applied voltage thereon. In various embodiments, the applied voltage on the ultrasonic transducer may be from about 0 V to about 50 V, where 0 V means that no ultrasonic wave is generated. In various embodiments, the applied voltage on the ultrasonic transducer may fall in a range with start and end points selected from the following group of numbers: 0, 5, 10, 15, 20, 25, 30, 35, 40, 45, and 50 V. In various embodiments, the ultrasonic transducer may be configured to generate ultrasound waves with varying intensities, thereby applying varying ultrasonic pressure levels on the material.

In various embodiments, the ultrasonic transducer, e.g., air-coupled ultrasonic transducer may comprise a single-element (i.e., non-phased array) transducer, e.g., single-element air-coupled transducer. In various embodiments, the ultrasonic transducer, e.g., air-coupled ultrasonic transducer may comprise a phased array comprising a plurality of ultrasonic/ultrasound transducers configured to generate directed ultrasonic waves to a 3D spatial location at the target region. In various embodiments, the phased array may have a focus point which is configured to coincide with a location on the target region of the material where the emergent beam is reflected and/or transmitted to be detected by the electromagnetic wave detector. In various embodiments, the ultrasonic transducer, e.g., air-coupled ultrasonic transducer may comprise a one-dimensional (1D) array of ultrasonic transducers elements. In various embodiments, the ultrasonic transducer, e.g., air-coupled ultrasonic transducer may comprise a two-dimensional (2D) array of ultrasonic transducers elements. In various embodiments, the air-coupled 2D phased array transducer may be made of Capacitive Micromachined Ultrasonic Transducers (CMUT), Piezoelectric Micromachined Ultrasonic Transducers (PMUT) or other transducer fabrication methods. In various embodiments, a phased array may advantageously allow an ultrasound beam to be created (having a given spatial distribution) and allow for electronically steering and focusing the beam in a target volume without the need for mechanical means to steer or reposition the transducer.

In various embodiments, the ultrasonic module may comprise an ultrasonic applicator in the form of a pulsed laser device. In various embodiments, the pulsed laser device is configured to emit pulses of electromagnetic waves (i.e., laser pulses) for applying/generating the ultrasonic pressure at the target region on the surface of the material. In various embodiments, the pulsed laser device may be coupled to a pulsed laser control/controller unit. The pulsed laser control may be configured to monitor and control the pulsed laser parameters such as pulse power (e.g., peak power, average power), pulse energy, pulse period, repetition rate, pulse width, pulse duration, laser wavelength. In various embodiments, a typical energy per pulse may range from about 0.1 mJ to about 100 mJ with a pulse duration in the range from about 0.1 ns to about 100 ns. In various embodiments, an area of laser excitation could be adjusted so that the laser power intensity is within the thermoacoustic regime of the testing materials.

In various embodiments, the pulsed laser device utilizes a laser-ultrasonics technique to generate ultrasonic waves. The physical principle is based on thermal expansion (also called thermoelastic regime) or ablation. In the thermoelastic regime, the ultrasound is generated by a sudden thermal expansion due to heating of a tiny surface of the material by the laser pulse. In the ablation regime, a plasma is often formed above the material surface and its expansion can make a substantial contribution to the ultrasonic generation. In various embodiments, the pulsed laser may be configured to operate in the thermoelastic regime.

In various embodiments, the pulsed laser device is configured to transmit the pulses of electromagnetic waves (i.e., laser pulses) over the air to the target region of the material, thereby exerting an ultrasonic pressure on the material. This advantageously allows the ultrasonic pressure to be exerted on the material in a non-contact manner. In various embodiments, the pulsed laser device may be configured such that laser pulses have an output that does not substantially damage the material. For example, the output of the pulsed laser (i.e., power and wavelength) may be adjusted such that it is capable of exerting the ultrasonic pressure on the material and yet does not substantially damage the material. This may render the pulsed laser device safe for use in in-vivo measurements, e.g., in-vivo measurement on the eye.

In various embodiments, the frequency content of the generated ultrasound may be determined by the frequency content of the laser pulses. In various embodiments, the pulsed laser device may be a nanosecond laser device, picosecond laser device or femtosecond laser device. In various embodiments, the pulsed laser may have a pulse duration in the order of nanoseconds, picoseconds, or femtoseconds. In various embodiments, the laser pulses may be diffused to cover the target region on the surface of the material.

In various embodiments, the ultrasonic pressure is controlled by the voltage applied to the transducers or the output power of the lasers. In various embodiments, the ultrasonic pressure applied on the material may be a substantially constant ultrasonic pressure without amplitude modulation. In various embodiments, the ultrasonic pressure applied on the material may be a varying ultrasonic pressure.

In various embodiments, the system or associated method disclosed herein may be employed to perform static measurement of the one or more mechanical properties of the material, where there is no synchronization between the ultrasonic pressure generation and electromagnetic wave detection/measurement. In various embodiments, a constant ultrasonic pressure may be applied for static measurement.

In various embodiments, the system or associated method disclosed herein may be employed to perform dynamic measurement of the one or more mechanical properties of the material. In various embodiments, dynamic measurement involves synchronization of the ultrasonic pressure generation and electromagnetic wave detection to capture the time delay or phase delay between the ultrasonic pressure generation and electromagnetic wave detection. In various embodiments, both pulse ultrasonic pressure generation and continuous ultrasonic pressure generation can be used with the dynamic measurement.

In various embodiments, the processing module is configured to communicate with the detection module to receive the emergent beam of electromagnetic waves. In various embodiments, the processing module (i.e., signal processing system) is configured to determine one or more measures corresponding to the one or more mechanical properties of the material, based on changes in the emergent beam of electromagnetic waves (i.e., detection signal). In various embodiments, the processing module is configured to perform time-domain spectrum measurement based on changes in the emergent beam of electromagnetic waves detected by the electromagnetic wave detector. In various embodiments, the processing module is configured to utilize amplitude change and phase change of the incident beam (e.g., incident THz radiation) and emergent beam (e.g., reflected/transmitted THz radiation) to perform analysis and calculation of the one or more mechanical properties of the material. In various embodiments, the processing module is configured to utilize one or more parameters of the ultrasonic pressure exerted, as input parameters for quantitatively calculating the one or more mechanical properties of the material.

For example, the processing module may be configured to detect the detection signal (e.g., THz signal) differences between a hydrogel with and without ultrasound pressure/stress (or with different ultrasound pressure/stress) measured by a THz detector. The changes in the THz signals reflect the changes of optical refraction index and deformation due to the presence of ultrasound pressure, which can be related to the mechanical properties of the hydrogels. The processing module may be further configured to process the detection signals to determine the measures of elasticity and rigidity in the hydrogel.

In various embodiments, the processing module may be configured to determine one or more measures corresponding to the one or more mechanical properties of the material by correlating (i) changes in one or more optical properties of the material (e.g. in relation to its effect on the THz wave detected), (ii) changes in one or more physical states of the material (e.g., deformation, hydration level), and/or (iii) level of ultrasonic pressure or changes in levels of ultrasonic pressures, in relation to changes in the emergent beam of electromagnetic waves received at the electromagnetic wave detector. In various embodiments, the processing module may further be configured to determine one or more measures corresponding to the one or more mechanical properties of the material by obtaining/generating a trend of the changes in (i) one or more optical properties of the material, and/or (ii) one or more physical states of the material (e.g., deformation, hydration level), in relation to changes in the emergent beam of electromagnetic waves received at the electromagnetic wave detector.

In various embodiments, the processing module may comprise an assembly of electronic components, micro-controllers, and computer. In various embodiments, the processing module may comprise an algorithm-embedded micro-controller, computer and/or electronics configured to determine/calculate the one or more mechanical properties of the material. In various embodiments, the processing module may be configured to execute an algorithm for analysing, determining, or calculating the one or more mechanical properties of the material (e.g., elastic and rigidity analysis of a hydrogel). In various embodiments, the algorithm may comprise a plurality of steps comprising at least one of the following steps of: calculating elasticity and rigidity of a material based on the detection signals (e.g., terahertz signals) before the application of ultrasound pressure/stress and with the application of ultrasound pressure/stress (or at a first ultrasound pressure/stress and at a second ultrasound pressure/stress different from the first, and optionally at other different pressure/stress); measuring deformation of the material (e.g., hydrogel) under the ultrasound stress force; calculating the elastic and/or rigidity constant of the material; and further detecting any abnormal material deformation.

In various embodiments, the processing module may be configured to execute an algorithm to calculate the one or more mechanical properties of the material. In various embodiments, the processing module may be configured to execute an algorithm comprising the following steps of:
1. projecting an incident beam of electromagnetic waves, e.g., incident THz signals into a surface of a material/specimen, e.g., hydrogel surface or cornea;
2. measuring an emergent beam of electromagnetic waves, e.g., emergent THz signals reflected from or transmitted through the specimen (i.e., measuring the specimen's THz reflection/transmission), prior to application of ultrasound pressure;
3. projecting ultrasound pressure on the surface of the specimen, e.g., hydrogel surface or cornea surface;
4. measuring an emergent beam of electromagnetic waves, e.g., emergent THz signals reflected from or transmitted through the specimen (i.e., measuring the specimen's THz reflection/transmission), while the surface of the specimen is subjected to ultrasonic pressure; and
5. performing an analysis algorithm e.g., elastic/rigidity analysis algorithms based on changes of optical refractive index and deformation.

When the specimen is under plane stress state, refractive index variations ΔN can be expressed using stress parameters according to the following equation:

$$Δ N = \frac{c ( {δ}_{1} - {δ}_{1}^{′} )}{2 π fd} = \frac{c Δ δ s}{2 π fd}$$

where ΔN represents changes in the optical refractive index measured by a THz time domain spectra, δ₁ and δ′₁ are phases of the emergent THz signals before and after the application of ultrasonic pressure, Δδₛ is the phase delay changes caused by stress, f is the frequency of the THz radiation, c is the speed of light in the vacuum and d is the original thickness of the specimen.

The phase delay of the emergent THz signal, e.g., THz pulse is mainly caused by two factors, one is the change in refractive index caused by the law of elastic effect and the other is the change in thickness caused by the Poisson's effect.

The thickness of the specimen changes under stress, and can be expressed as

$$Δ d = \frac{d \cdot μ \cdot σ}{E}$$

where μ, σ, and E are Poisson's ratio, interior tensile stress, and elastic modulus, respectively. The thickness change can be observed from the time domain spectra. Supposing the initial refractive index of the specimen is N₀, the phase change of Δδ_{d} induced by the decrease of thickness can be written as follows:

$$Δ {δ}_{d} = \frac{2 π f ( {N}_{0} - 1 ) Δ d}{c}$$

The phase change δδ measured during operation of the system for non-contact measurement has two parts, Δδₛ and Δδ_{d}:

$$Δ δ = Δ {δ}_{s} + Δ {δ}_{d}$$

Therefore, the phase change induced by the stress can be further corrected as:

$$Δ N = \frac{c}{2 π d} \frac{Δδ}{f} + \frac{( {N}_{0} - 1 ) μ σ}{E}$$

Accordingly, in various embodiments, the relationship between the phase changes and refractive index changes can be obtained from THz time domain spectroscopy. In various embodiments, a fitting curve may be plotted and the Young's modulus of the specimen may be calculated from the intercept and slope of the fitting curve. It will be appreciated that the associated method of non-contact measurement disclosed herein may be employed to execute the algorithm as described herein.

### Method of Non-Contact Measurement of One or More Mechanical Properties of a Material

In various embodiments, there is provided a method of non-contact measurement of one or more mechanical properties of a material, the method comprising, emitting an incident beam of electromagnetic waves from an electromagnetic wave emitter towards a target region of the material; applying or varying an ultrasonic pressure on the target region of the material; detecting an emergent beam of electromagnetic waves reflected from the target region and/or transmitted through the target region with an electromagnetic wave detector; and determining one or more measures corresponding to the one or more mechanical properties with a processing module, based on changes in the emergent beam of electromagnetic waves (e.g. due to the application or variation of ultrasonic pressure on the target region which may cause a perturbation of the material/target region).

In various embodiments, the method may be performed using the system for non-contact measurement as disclosed herein. In various embodiments, the material may comprise a hydrogel or a soft tissue, e.g., cornea tissue, in an eye of a mammalian subject. In various embodiments, the one or more mechanical properties may include but are not limited to elasticity, rigidity, viscoelasticity and rheology. In various embodiments, the measures of mechanical properties may include but are not limited to a modulus of elasticity (i.e., Young's modulus and shear modulus), a modulus of rigidity (i.e., stiffness), viscoelastic properties (i.e., dynamic storage and loss moduli) and rheology properties (e.g., relaxation times and compliance). In various embodiments, the incident beam of electromagnetic waves emitted by the electromagnetic wave emitter has a frequency falling in a range from 0.1 THz to 10 THz. In various embodiments, the emergent beam of electromagnetic waves emitted by the electromagnetic wave emitter has a frequency falling in a range from 0.1 THz to 10 THz. In various embodiments, the ultrasonic pressure applied at the target region on the surface of the material is substantially constant without amplitude modulations.

In various embodiments, varying the ultrasonic pressure on the target region of the material may comprise varying a voltage that is applied on an ultrasonic transducer or an output power of a pulsed laser device. In various embodiments, the ultrasound waves generated by the ultrasonic transducer may have an intensity that is proportional to the applied voltage. In various embodiments, varying the ultrasonic pressure on the target region of the material may comprise varying the applied voltage from about 0 V to about 50 V, where 0 V means that no ultrasonic wave is generated.

In various embodiments, the step of applying or varying the ultrasonic pressure on the target region of the material is configured to generate/produce a change in one or more properties of the material. The one or more properties of the material may include but are not limited to optical property (e.g., refraction index) and mechanical property (e.g., deformation). In various embodiments, the step of applying or varying the ultrasonic pressure on the target region of the material is configured to generate/produce stress at the target region of the material. For example, applying or varying the ultrasonic pressure on the material may produce mechanical deformation of the material, said mechanical deformation resulting in a change in one or more characteristics of the emergent beam detected by the electromagnetic wave detector. For example, applying or varying the ultrasonic pressure applied on the material may produce a change in the refractive index of the material, said change in refractive index resulting in a change in one or more characteristics of the emergent beam detected by the electromagnetic wave detector. In various embodiments, the step of applying or varying the ultrasonic pressure on the target region of the material may produce a continuous or pulsed vibration at a specific frequency in the material which leads to dynamic changes in the refractive index at the said specific frequency, which in turn result in a change in one or more characteristics of the emergent beam detected by the electromagnetic wave detector. In various embodiments, the step of applying or varying the ultrasonic pressure on the target region of the material may be synchronized with the detection of electromagnetic waves. With synchronization between the ultrasonic generation and electromagnetic wave detection, phase delay between the applied ultrasonic pressure and the electromagnetic wave could be quantified and related to the viscoelastic and rheology properties at the said frequency.

In various embodiments, the changes in the emergent beam of electromagnetic waves are determined with respect to a reference emergent beam detected at a different time point (or based on a predetermined reference emergent beam and properties thereof). In various embodiments, the method comprises performing time-domain spectroscopy/time-domain spectrum measurement, e.g., terahertz time-domain spectroscopy, based on the emergent beam of electromagnetic waves (i.e., detection signals) detected by the electromagnetic wave detector before and after application of the ultrasonic pressure (or at different ultrasound pressure/stress applied).

In various embodiments, the method comprises emitting/projecting the incident beam of electromagnetic waves (e.g., incident THz signal) into a surface of the material (e.g., hydrogel) at the target region; measuring the emergent beam of electromagnetic waves (e.g., emergent THz signal) reflected from the target region or transmitted through the target region; generating and projecting ultrasonic pressure on the surface of the material; measuring the emergent beam of electromagnetic waves (e.g., emergent THz signal) reflected from the target region or transmitted through the target region under the ultrasonic pressure; and performing an analysis algorithm (e.g., elasticity/rigidity analysis algorithm) to generate measures of the one or more mechanical properties (e.g., elasticity and rigidity).

In various embodiments, the method comprises (i) emitting the incident beam of electromagnetic waves and detecting the emergent beam of electromagnetic waves at a first time point; (ii) applying or varying the ultrasonic pressure on the target region of the material; (iii) emitting the incident beam of electromagnetic waves and detecting the emergent beam of electromagnetic waves at a second time point; (iv) optionally repeating steps (i) to (iii) one or more times; and (v) determining the one or more measures corresponding to the one or more mechanical properties based on differences in emergent beams at at least two different time points. This may advantageously allow monitoring of time-dependent changes of the one or more mechanical properties of the material.

In various embodiments, the step of applying or varying the ultrasonic pressure on the target region of the material comprises applying or varying the ultrasonic pressure over the air, e.g., using air-transmitted ultrasonic waves or air-transmitted laser pulses. In various embodiments, the ultrasonic pressure is applied using an air-coupled ultrasonic transducer over air. In various embodiments, the ultrasonic pressure is applied using a pulsed laser device over air, by applying pulses of electromagnetic waves (i.e., laser pulses) having a pulse duration in the order of nanoseconds, picoseconds, or femtoseconds. In various embodiments, the pulses of electromagnetic waves are diffused to cover the target region on the surface of the material. In other words, there may be no physical contact between the air-coupled ultrasonic transducer and pulsed laser device with the material.

In various embodiments, the step of determining one or more measures corresponding to the one or more mechanical properties of the material may comprise correlating (i) changes in one or more optical properties of the material (e.g., in relation to its effect on the THz wave detected), (ii) changes in one or more physical states of the material (e.g., deformation, hydration level) and/or (iii) level of ultrasonic pressure or changes in levels of ultrasonic pressures, in relation to changes in the emergent beam of electromagnetic waves received at the electromagnetic wave detector. In various embodiments, the step of determining one or more measures corresponding to the one or more mechanical properties of the material may further comprise obtaining a trend of the changes in (i) one or more optical properties of the material, and/or (ii) one or more physical states of the material (e.g., deformation, hydration level), in relation to changes in the emergent beam of electromagnetic waves received at the electromagnetic wave detector. In various embodiments, the step of determining one or more measures corresponding to the one or more mechanical properties of the material may comprise utilizing one or more parameters of the ultrasonic pressure exerted, as input parameters for quantitatively calculating the one or more mechanical properties of the material.

In various embodiments, the step of determining one or more measures corresponding to the one or more mechanical properties of the material may comprise performing an analysis algorithm to determine the one or more measures corresponding to the one or more mechanical properties. In various embodiments, the algorithm may comprise a plurality of steps comprising at least one of the following steps of: calculating elasticity and rigidity of a material based on the detection signals (e.g., terahertz signals) before the application of ultrasound pressure/stress and with the application of ultrasound pressure/stress; measuring changes in optical properties (e.g., refractive index) under the ultrasound stress force; measuring changes in state (e.g., deformation) of the material (e.g., hydrogel) under the ultrasound stress force; measuring changes in hydration levels of the material; correlating changes in optical properties (e.g., refractive index), state (e.g., deformation) and/or hydration level with changes in the emergent beam of electromagnetic waves (e.g., emergent THz signals); calculating the elastic and/or rigidity constant of the material; and further detecting any abnormal material deformation.

In various embodiments, there is provided a computer readable storage medium having stored thereon instructions for instructing a processing unit of a system to execute a method of non-contact measurement of one or more mechanical properties of a material in accordance with one or more embodiments of the method disclosed herein. For example, the instructions may include to execute a method comprising, emitting an incident beam of electromagnetic waves from an electromagnetic wave emitter towards a target region of the material; applying or varying an ultrasonic pressure on the target region of the material; detecting an emergent beam of electromagnetic waves reflected from the target region and/or transmitted through the target region with an electromagnetic wave detector; and determining one or more measures corresponding to the one or more mechanical properties with a processing module, based on changes in the emergent beam of electromagnetic waves. In some embodiments, the computer readable storage medium is a non-transitory computer storage medium.

### BRIEF DESCRIPTION OF FIGURES

FIG. 1 is a schematic diagram of a system for non-contact measurement of one or more mechanical properties of a material, e.g., hydrogel, in an example embodiment.

FIG. 2 is a schematic diagram of a system for non-contact measurement of one or more mechanical properties of a material, e.g., hydrogel, in another example embodiment.

FIG. 3 is a schematic diagram of a phased array in an example embodiment.

FIG. 4 is a schematic diagram of a system for non-contact measurement of one or more mechanical properties of a material, e.g., hydrogel, in yet another example embodiment.

FIG. 5 is a graph showing changes of emergent/return THz signals on hydrogel with different driving voltages of air-coupled transducer in an example embodiment.

FIG. 6 is a graph showing changes of THz signals with the hydration level of hydrogel in an example embodiment.

FIG. 7 is a graph showing THz time domain spectra collected on the surface of two hydrogel samples under test in an example embodiment.

FIG. 8 is a graph showing phase change/difference versus frequency for two hydrogel samples under test in an example embodiment. The phase versus frequency before and after ultrasound mechanical stress are shown in the inset graph of FIG. 8.

FIG. 9 is a graph showing refractive index changes versus frequency for two hydrogel samples under test in an example embodiment. The refractive index values of the two hydrogels are shown in the inset graph of FIG. 9.

FIG. 10 is a graph showing refractive index difference versus phase difference for two hydrogel samples under test in an example embodiment.

FIG. 11 is a schematic flow chart for illustrating a method of rigidity/elastic analysis of a hydrogel in an example embodiment.

FIG. 12 is a schematic drawing of a computer system suitable for implementing an example embodiment.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

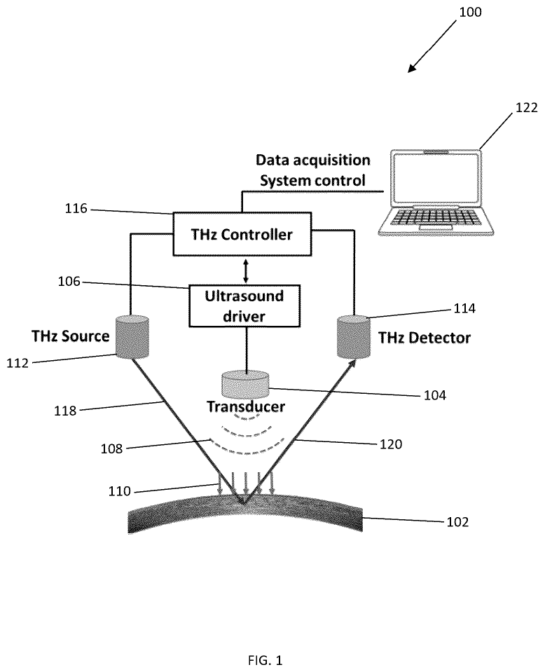

FIG. 1 is a schematic diagram of a system 100 for non-contact measurement of one or more mechanical properties of a material, e.g., hydrogel 102, in an example embodiment. The one or more mechanical properties of the material may be elastic properties of the hydrogel 102.

The system 100 comprises an ultrasonic module, said ultrasonic module comprising an ultrasonic applicator, e.g., an air-coupled ultrasound transducer 104, coupled to an ultrasound driver 106 including a function generator and an amplifier. The air-coupled ultrasound transducer 104 is configured to emit ultrasonic waves 108 to exert ultrasonic pressure 110 on a target region of the hydrogel 102. The ultrasonic applicator may be a single-element (non-phased array) transducer or a phased array comprising a plurality of ultrasonic transducers.

The system 100 further comprises a detection module, said detection module comprising an electromagnetic wave emitter, e.g., a THz source/emitter 112, and an electromagnetic wave detector, e.g., a THz detector 114, each coupled to a controller, e.g., THz controller 116. The THz controller 116 is in electrical communication with the ultrasound driver 106. The THz source 112 serves as a signal emitter and is configured to emit an incident beam of electromagnetic waves, e.g., THz signals 118, towards the target region of the hydrogel 102. The THz detector 114 serves as a signal receiver and is configured to detect/receive an emergent beam of electromagnetic waves, e.g., THz signals 120 reflected from the target region of the hydrogel 102. The detection module utilizes THz time domain spectroscopy to measure deformation and changes of refraction index in the hydrogel 102.

The system 100 further comprises a processing module, e.g., signal processing unit 122 comprising software and computer hardware. The signal processing unit 122 is configured to determine one or more measures, e.g., elastic constant corresponding to the elasticity of the hydrogel 102, based on changes in the THz signals 120.

In the example embodiment, the system 100 is configured in a reflection mode. The air-coupled ultrasound transducer 104, THz emitter 112, and THz detector 114 are positioned on the same side of the hydrogel 102, such that the THz detector 114 detects the THz signals 120 reflected from the target region of the hydrogel 102. During operation, the air-coupled ultrasound transducer 104 provides sound pressure (i.e., ultrasonic pressure 110) on the hydrogel 102. The THz emitter 112 generates terahertz radiation which is reflected from a surface of the hydrogel 102. The THz detector 114 receives the THz signals 120 which are reflected from the surface of the hydrogel 102 before and after the sound pressure is applied on the surface of the hydrogel 102. The signal processing unit 122 is used to calculate the elastic constant of the hydrogel 102 by utilizing the deformation and changes of refraction index measured by the THz time domain spectroscopy.

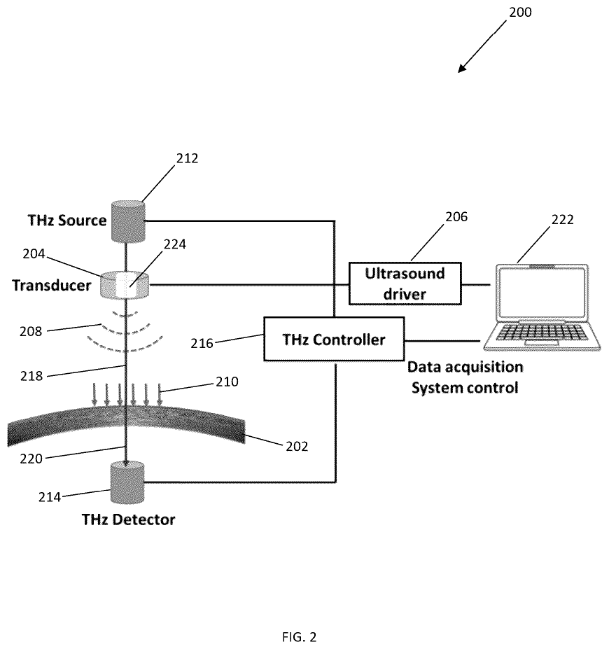

FIG. 2 is a schematic diagram of a system 200 for non-contact measurement of one or more mechanical properties of a material, e.g., hydrogel 202, in another example embodiment. The one or more mechanical properties of the material may be elastic properties of the hydrogel 202.

The system 200 comprises an ultrasonic module, said ultrasonic module comprising an ultrasonic applicator, e.g., an air-coupled ultrasound transducer 204, coupled to an ultrasound driver 206 including a function generator and an amplifier. The air-coupled ultrasound transducer 204 is configured to emit ultrasonic waves 208 to exert ultrasonic pressure 210 on a target region of the hydrogel 202. The ultrasonic applicator may be a single-element (non-phased array) transducer or a phased array comprising a plurality of ultrasonic transducers.

The system 200 further comprises a detection module, said detection module comprising an electromagnetic wave emitter, e.g., a THz source/emitter 212, and an electromagnetic wave detector, e.g., a THz detector 214, each coupled to a controller, e.g., THz controller 216. The THz source 212 serves as a signal emitter and is configured to emit an incident beam of electromagnetic waves, e.g., THz signals 218, towards the target region of the hydrogel 202. The THz detector 214 serves as a signal receiver and is configured to detect/receive an emergent beam of electromagnetic waves, e.g., THz signals 220 reflected from the target region of the hydrogel 202. The detection module utilizes THz time domain spectroscopy to measure deformation and changes of refraction index in the hydrogel 202.

The system 200 further comprises a processing module, e.g., signal processing unit 222 comprising software and computer hardware. The signal processing unit 222 is configured to determine one or more measures, e.g., elastic constant corresponding to the elasticity of the hydrogel 202, based on changes in the THz signals 220.

In the example embodiment, the system 200 is configured in a transmission mode. The air-coupled ultrasound transducer 204 and THz emitter 212 are positioned on a first (top) side of the hydrogel 202, and the THz detector 214 is positioned on a second (bottom) opposite side of the hydrogel 202, such that the THz detector 214 detects the THz signals 220 transmitted through the target region of the hydrogel 202. During operation, the air-coupled ultrasound transducer 204 provides sound pressure (i.e., ultrasonic pressure 210) on the hydrogel 202. The THz emitter 212 generates terahertz radiation which passes through the hydrogel 202. The THz detector 214 receives the THz signals 220 which pass through the hydrogel 202 before and after the sound pressure is applied on the surface of the hydrogel 202. The signal processing unit 222 is used to calculate the elastic constant of the hydrogel 202 by utilizing the deformation and changes of refraction index measured by the THz time domain spectroscopy. As shown in FIG. 2, the THz beam 218 is substantially perpendicular to the surface of the hydrogel 102 such that maximum THz sensitivity could be achieved. To facilitate the transmission of the THz signal 218, the centre of the air-coupled ultrasound transducer 204 may have a cavity 224 for THz signal 218 to pass through.

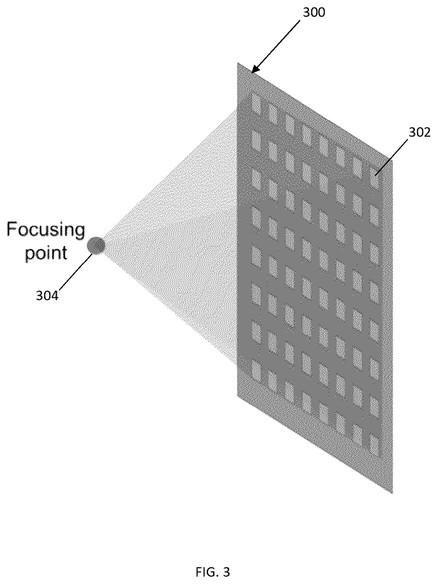

FIG. 3 is a schematic diagram of a phased array 300 in an example embodiment. The phased array 300 comprises a plurality of transducer elements 302. The phased array 300 may be configured in a 1D or 2D arrangement such that it can generate controllable ultrasonic waves in an arbitrary spatial location in the material, e.g., hydrogel samples. The phased array 300 made of multiple elements may be utilized in the air-coupled transducers 104 and 204 of FIGS. 1 and 2 as an air-coupled 2D phased array transducer. In the example embodiment, the location of the THz measurement is configured to coincide with the focus point 304 of the phased array air-coupled transducer. The air-coupled 2D phased array transducer may be made of Capacitive Micromachined Ultrasonic Transducers (CMUT), Piezoelectric Micromachined Ultrasonic Transducers (PMUT) or other transducer fabrication methods.

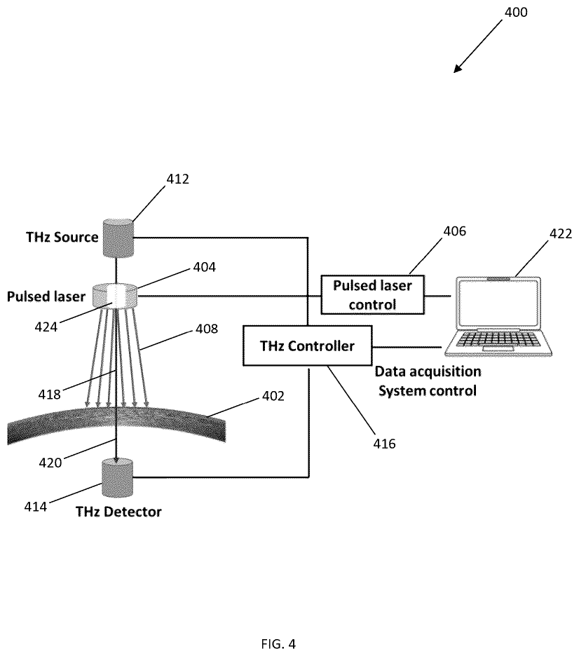

FIG. 4 is a schematic diagram of a system 400 for non-contact measurement of one or more mechanical properties of a material, e.g., hydrogel 402, in yet another example embodiment. The one or more mechanical properties of the material may be elastic properties of the hydrogel 402.

The system 400 comprises an ultrasonic module, said ultrasonic module comprising an ultrasonic applicator, e.g., a pulsed laser 404 coupled to a pulsed laser control 406. The pulsed laser 404 is configured to emit electromagnetic waves to exert ultrasonic pressure on a target region of the hydrogel 402. The system 400 of FIG. 4 differs from the system 100 of FIG. 1 and the system 200 of FIG. 2 in that the ultrasonic pressure is generated by a pulsed laser. The pulsed laser utilizes a laser-ultrasonics technique using lasers to generate ultrasonic waves. The physical principle is based on thermal expansion (also called thermoelastic regime) or ablation. In the thermoelastic regime, the ultrasound is generated by a sudden thermal expansion due to heating of a tiny surface of the material by the laser pulse. The pulsed laser may be a nano-second laser, a pico-second laser or a femtosecond laser. The laser may be diffused to cover a sizable area in the target region of the hydrogel 402.

The system 400 further comprises a detection module, said detection module comprising an electromagnetic wave emitter, e.g., a THz source/emitter 412, and an electromagnetic wave detector, e.g., a THz detector 414, each coupled to a controller, e.g., THz controller 416. The THz source 412 serves as a signal emitter and is configured to emit an incident beam of electromagnetic waves, e.g., THz signals 418, towards the target region of the hydrogel 402. The THz detector 414 serves as a signal receiver and is configured to detect/receive an emergent beam of electromagnetic waves, e.g., THz signals 420 reflected from the target region of the hydrogel 402. The detection module utilizes THz time domain spectroscopy to measure deformation and changes of refraction index in the hydrogel 402.

The system 400 further comprises a processing module, e.g., signal processing unit 422 comprising software and computer hardware. The signal processing unit 422 is configured to determine one or more measures, e.g., elastic constant corresponding to the elasticity of the hydrogel 402, based on changes in the THz signals 420.

In the example embodiment, the system 400 is configured in a transmission mode. The pulsed laser 404 and THz emitter 412 are positioned on a first (top) side of the hydrogel 402, and the THz detector 414 is positioned on a second (bottom) opposite side of the hydrogel 402, such that the THz detector 414 detects the THz signals 420 transmitted through the target region of the hydrogel 402. During operation, the pulsed laser 404 generates ultrasonic pressure on the hydrogel 402. The THz emitter 412 generates terahertz radiation which passes through the hydrogel 402. The THz detector 414 receives the THz signals 420 which pass through the hydrogel 402 before and after the ultrasonic pressure is applied on the surface of the hydrogel 402. The signal processing unit 422 is used to calculate the elastic constant of the hydrogel 402 by utilizing the deformation and changes of refraction index measured by the THz time domain spectroscopy. As shown in FIG. 4, the THz beam 418 is substantially perpendicular to the surface of the hydrogel 402 such that maximum THz sensitivity could be achieved. To facilitate the transmission of the THz signal 418, the centre of the pulsed laser 404 may be substantially optically transparent to THz signal e.g., such as having a cavity/opening 424 for THz signal 418 to pass through.

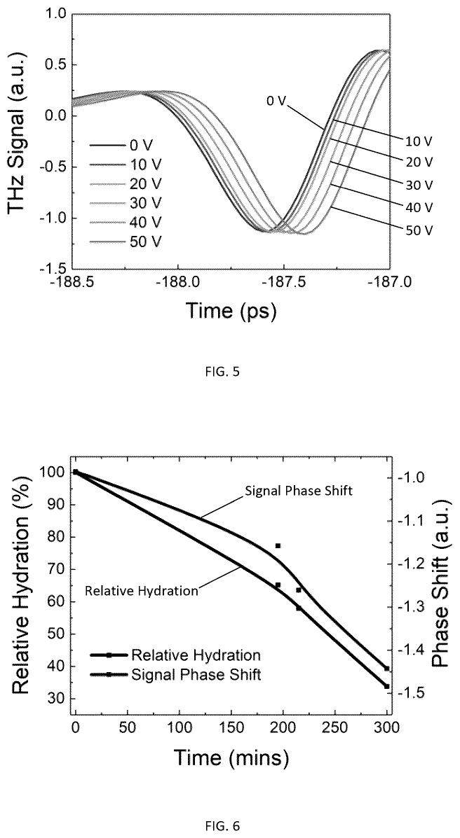

FIG. 5 is a graph showing changes of emergent/return THz signals on hydrogel with different driving voltages of air-coupled transducer in an example embodiment. Changes of THz signals are investigated when different amplitudes of ultrasound signals are applied on a hydrogel sample. The ultrasound intensity generated by the air-coupled ultrasound transducer is proportional to that of applied voltage. As shown in FIG. 5, with the increase of the ultrasound intensity applied on the hydrogel sample, the THz signals show a monotonic increase in time delay. The time delays at different ultrasound pressure levels can be used in an algorithm to calculate the elastic properties of the hydrogels.

FIG. 6 is a graph showing changes of THz signals with the hydration level of hydrogel in an example embodiment. A hydrogel sample was placed in air and dehydrated at elevated temperature. Table 1 lists the changes of mass, thickness, and relative hydration levels over time. The measurement was taken at different times corresponding to different hydration levels. FIG. 6 shows the measurement results of the hydrogel sample at different hydration levels. As shown in FIG. 6, the changes of THz signals correlate well with the changes of hydration levels. In addition, as shown in the example embodiments of FIG. 1 to FIG. 4, the system and method of using the system to perform measurement is fully non-contact.

TABLE 1
Mass, thicknesses, relative hydration level
of hydrogel sample with dehydration over time

| | | | Thickness | Relative |
|---|---|---|---|---|
| Measurement | Time (min) | Mass (g) | (mm) | Hydration Level |
| 1 | 0 | 6.90 | 3.05 | 100.00 |
| 2 | 195 | 4.50 | 2.20 | 65.22 |
| 3 | 215 | 4.00 | 2.00 | 57.97 |
| 4 | 230 | 3.50 | 1.80 | 50.72 |
| 5 | 300 | 2.33 | 0.80 | 33.77 |

Therefore, it is feasible to use this technology to monitor the changes of elastic properties due to the changes of hydration levels in a non-contact manner. One of the applications of the technique is to monitor changes of elastic properties of hydrogels due to changes in hydration levels over time. In clinical practices, it is also of interest to know the hydration levels of soft tissues such as cornea at different dates and times.

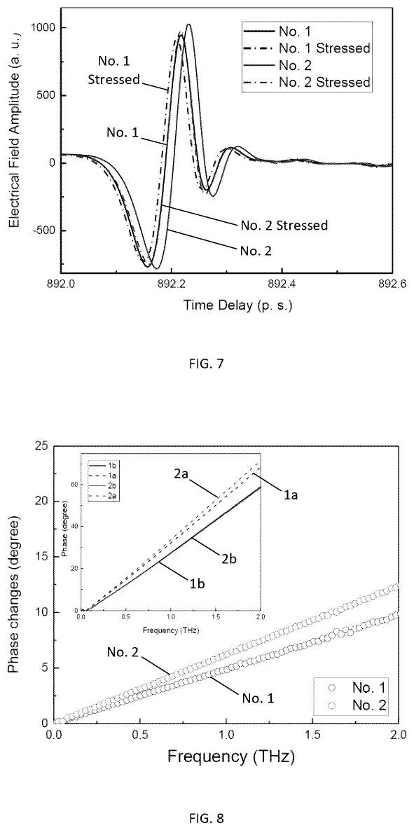

In the following example embodiments described with reference to FIG. 7 to FIG. 10, ultrasound mechanical stress was applied to generate mechanical deformation of hydrogel samples. Thereafter, a correlation between phase change and refractive index was used to derive the Young's modulus of the hydrogel samples. The results described with reference to FIG. 7 to FIG. 10 were obtained using an example embodiment of the system as disclosed herein that is configured in the reflection mode. It will be appreciated that example embodiments of the system as disclosed herein may be configured in transmission mode or a combination of reflection and transmission modes to perform non-contact measurement of one or more mechanical properties of a material. It will be appreciated that the measurements may be performed on other samples such as the cornea of a subject.

FIG. 7 shows the THz time domain spectra collected on the surface of two hydrogel samples with and without ultrasound mechanical stress. The two hydrogels have similar chemical composition and different hydration levels. Sample No. 1 has a higher hydration level. The solid curves show the emergent THz signal before ultrasound mechanical stress, while the dash lines show the emergent THz signal while the hydrogel samples are under ultrasound mechanical stress. When the ultrasound mechanical stress is on, the curves shifted and the reflection intensity slightly reduced. Hydrogels with different hydration levels under the same ultrasound mechanical stress show different levels of response. The higher changes of THz signal reflect lower rigidity of the hydrogel samples. The measurement has been repeated several times and the results are repeatable.

FIG. 8 shows the phase change/difference versus frequency for two hydrogel samples with and without ultrasound mechanical stress, their phase versus frequency before and after the ultrasound mechanical stress are shown in the inset figure. The solid lines show the phases before ultrasound mechanical stress, and the dash lines show the phases while the hydrogel samples are under ultrasound mechanical stress. The results demonstrated that differences in hydration levels contribute to changes of optical and mechanical properties of the hydrogel, which in turn lead to time-domain phase changes.

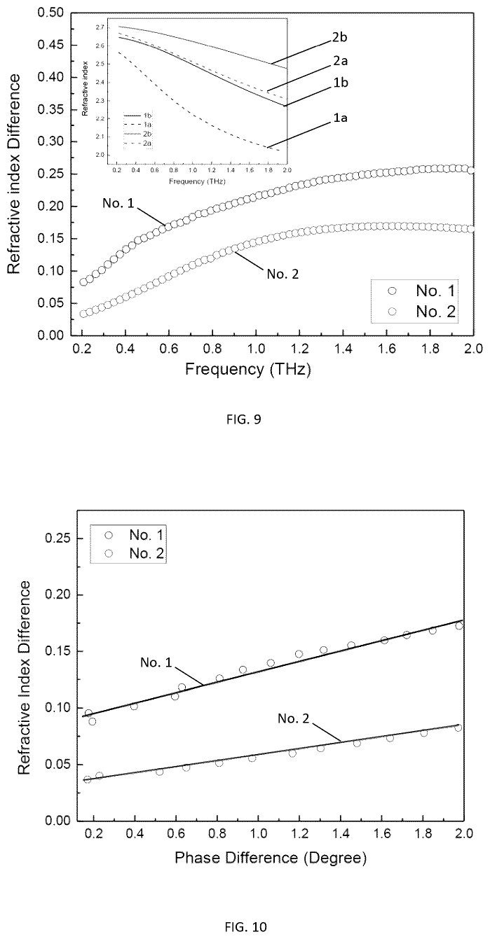

FIG. 9 shows the refractive index changes versus frequency for two hydrogel samples with and without ultrasound mechanical stress. Information on amplitude and phase could be obtained from time domain THz spectroscopy, and therefore the optical and electrical parameters could be calculated. Due to the different hydration levels of the two hydrogel samples, their THz time domain spectra demonstrated different behavior, the calculated refractive index and the refractive index difference due to stress also shows different behaviour. These results demonstrated that the mechanical properties of hydrogels are different at different hydration levels.

FIG. 10 shows the refractive index difference versus phase difference for two hydrogel samples under test. Sample No. 1 has a higher hydration level. The hydrogel sample with the higher value and higher slope indicates that with the same phase change, the refractive index shift induced is much higher. Linear fitting is done for refractive index difference versus phase difference in order to calculate the parameters according to the following equation:

$$Δ N = \frac{c}{2 π d} \frac{Δδ}{f} + \frac{( {N}_{0} - 1 ) μ σ}{E}$$

The fitting parameters include the slope and the intercept. From the above formulas, the Young's module and the hydrogel thickness can be calculated.

The refractive index difference and phase difference may be obtained by performing the following steps:
1. projecting an incident beam of electromagnetic waves, e.g., incident THz signals into the surface of the hydrogel sample;
2. measuring an emergent beam of electromagnetic waves, e.g., emergent THz signals reflected from or transmitted through the hydrogel sample prior to application of ultrasound pressure;
3. projecting ultrasound pressure on the hydrogel surface;
4. measuring the emergent THz signals reflected from or transmitted through the hydrogel sample while the surface of the hydrogel sample is subjected to ultrasonic pressure; and
5. performing elastic/rigidity analysis algorithms based on changes of optical refractive index and deformation.

When the hydrogel sample is under plane stress state, refractive index variations ΔN can be expressed using stress parameters according to the following equation:

$$Δ N = \frac{c ( {δ}_{1} - {δ}_{1}^{′} )}{2 π fd} = \frac{c Δ δ s}{2 π fd}$$

where ΔN represents changes in the optical refractive index measured by a THz time domain spectra, δ₁ and δ′₁ are phases of the emergent THz signals before and after the application of ultrasonic pressure, Δδₛ is the phase delay changes caused by stress, f is the frequency of the THz radiation, c is the speed of light in the vacuum and d is the original thickness of the hydrogel sample.

The phase delay of the emergent THz signal, e.g., THz pulse is mainly caused by two factors, one is the change in refractive index caused by the law of elastic effect and the other is the change in thickness caused by the Poisson's effect.

The thickness of the hydrogel sample changes under stress, which can be expressed as

$$Δ d = \frac{d \cdot μ \cdot σ}{E}$$

where μ, σ, and E are Poisson's ratio, interior tensile stress, and elastic modulus, respectively. The thickness change can be observed from the time domain spectra. Supposing the initial refractive index of the hydrogel sample is N₀, the phase change of Δδ_{d} induced by the decrease of thickness can be written as follows:

$$Δ {δ}_{d} = \frac{2 π f ( {N}_{0} - 1 ) Δ d}{c}$$

The phase change δδ measured in the experiment has two parts, Δδₛ and Δδ_{d}:

$$Δ δ = Δ {δ}_{s} + Δ {δ}_{d}$$

Therefore, the phase change induced by the stress can be further corrected as:

$$Δ N = \frac{c}{2 π d} \frac{Δδ}{f} + \frac{( {N}_{0} - 1 ) μ σ}{E}$$

From THz time domain spectroscopy, the relationship between the phase changes and refractive index changes can be obtained, and from the fitting curve intercept and slope, the Young's modulus can be calculated.

Table 2 shows the Young's modulus and average Young's modulus of the hydrogel samples derived from the slopes and intercepts of the graph of FIG. 10.

TABLE 2
Derivation of Young's modulus and average Young's modulus
of hydrogel samples from the slopes and intercepts of graphs

| | | | Young's modulus | Young's modulus |
|---|---|---|---|---|
| | Slope | Intercept | (MPa) | (average) (MPa) |
| 1 | 0.01983 | 0.04597 | 0.14847 | 0.15543 |
| | 0.01831 | 0.04458 | 0.1531 | |
| | 0.01845 | 0.04143 | 0.16474 | |
| 2 | 0.01699 | 0.0363 | 0.21694 | 0.20398 |
| | 0.01711 | 0.0334 | 0.20434 | |
| | 0.0176 | 0.0358 | 0.19064 | |

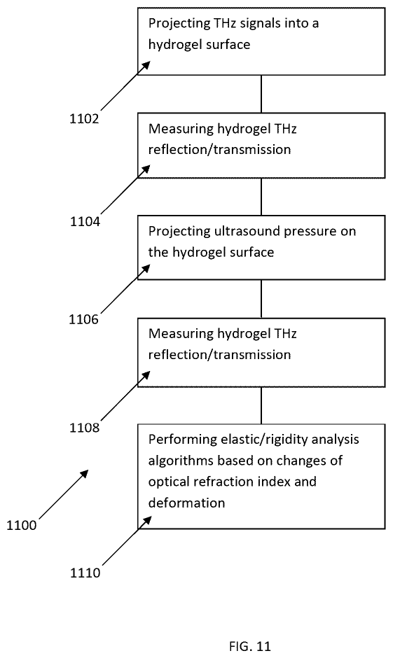

FIG. 11 is a schematic flow chart 1100 for illustrating a method of rigidity/elastic analysis of a hydrogel in an example embodiment. At step 1102, THz signals are projected onto a hydrogel surface. At step 1104, reflection/transmission of THz signals on the hydrogel are measured. At step 1106, ultrasound pressure is projected/applied on the hydrogel surface. At step 1108, reflection/transmission of THz signals on the hydrogel are measured. At step 1110, elasticity/rigidity analysis algorithms are performed based on changes of optical refraction index and deformation.

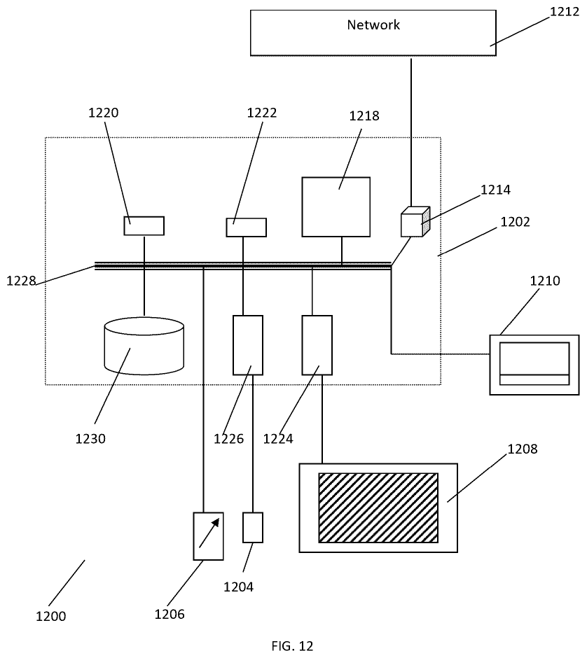

FIG. 12 is a schematic drawing of a computer system 1200 suitable for implementing an example embodiment. Different example embodiments can be implemented in the context of data structure, program modules, program and computer instructions executed in a computer implemented environment. A general purpose computing environment is briefly disclosed herein. One or more example embodiments may be embodied in one or more computer systems, such as is schematically illustrated in FIG. 12.

One or more example embodiments may be implemented as software, such as a computer program being executed within a computer system 1200, and instructing the computer system 1200 to conduct a method of an example embodiment.

The computer system 1200 comprises a computer unit 1202, input modules such as a keyboard 1204 and a pointing device 1206 and a plurality of output devices such as a display 1208, and printer 1210. A user can interact with the computer unit 1202 using the above devices. The pointing device can be implemented with a mouse, track ball, pen device or any similar device. One or more other input devices (not shown) such as a joystick, game pad, satellite dish, scanner, touch sensitive screen or the like can also be connected to the computer unit 1202. The display 1208 may include a cathode ray tube (CRT), liquid crystal display (LCD), field emission display (FED), plasma display or any other device that produces an image that is viewable by the user.

The computer unit 1202 can be connected to a computer network 1212 via a suitable transceiver device 1214, to enable access to e.g., the Internet or other network systems such as Local Area Network (LAN) or Wide Area Network (WAN) or a personal network. The network 1212 can comprise a server, a router, a network personal computer, a peer device or other common network node, a wireless telephone or wireless personal digital assistant. Networking environments may be found in offices, enterprise-wide computer networks and home computer systems etc. The transceiver device 1214 can be a modem/router unit located within or external to the computer unit 1202, and may be any type of modem/router such as a cable modem or a satellite modem.

It will be appreciated that network connections shown are exemplary and other ways of establishing a communications link between computers can be used. The existence of any of various protocols, such as TCP/IP, Frame Relay, Ethernet, FTP, HTTP and the like, is presumed, and the computer unit 1202 can be operated in a client-server configuration to permit a user to retrieve web pages from a web-based server. Furthermore, any of various web browsers can be used to display and manipulate data on web pages.

The computer unit 1202 in the example comprises a processor 1218, a Random Access Memory (RAM) 1220 and a Read Only Memory (ROM) 1222. The ROM 1222 can be a system memory storing basic input/output system (BIOS) information. The RAM 1220 can store one or more program modules such as operating systems, application programs and program data.

The computer unit 1202 further comprises a number of Input/Output (I/O) interface units, for example I/O interface unit 1224 to the display 1208, and I/O interface unit 1226 to the keyboard 1204. The components of the computer unit 1202 typically communicate and interface/couple connectedly via an interconnected system bus 1228 and in a manner known to the person skilled in the relevant art. The bus 1228 can be any of several types of bus structures including a memory bus or memory controller, a peripheral bus, and a local bus using any of a variety of bus architectures.

It will be appreciated that other devices can also be connected to the system bus 1228. For example, a universal serial bus (USB) interface can be used for coupling a video or digital camera to the system bus 1228. An IEEE 1394 interface may be used to couple additional devices to the computer unit 1202. Other manufacturer interfaces are also possible such as FireWire developed by Apple Computer and i. Link developed by Sony. Coupling of devices to the system bus 1228 can also be via a parallel port, a game port, a PCI board or any other interface used to couple an input device to a computer. It will also be appreciated that, while the components are not shown in the figure, sound/audio can be recorded and reproduced with a microphone and a speaker. A sound card may be used to couple a microphone and a speaker to the system bus 1228. It will be appreciated that several peripheral devices can be coupled to the system bus 1228 via alternative interfaces simultaneously.

An application program can be supplied to the user of the computer system 1200 being encoded/stored on a data storage medium such as a CD-ROM or flash memory carrier. The application program can be read using a corresponding data storage medium drive of a data storage device 1230. The data storage medium is not limited to being portable and can include instances of being embedded in the computer unit 1202. The data storage device 1230 can comprise a hard disk interface unit and/or a removable memory interface unit (both not shown in detail) respectively coupling a hard disk drive and/or a removable memory drive to the system bus 1228. This can enable reading/writing of data. Examples of removable memory drives include magnetic disk drives and optical disk drives. The drives and their associated computer-readable media, such as a floppy disk provide nonvolatile storage of computer readable instructions, data structures, program modules and other data for the computer unit 1202. It will be appreciated that the computer unit 1202 may include several of such drives. Furthermore, the computer unit 1202 may include drives for interfacing with other types of computer readable media.

The application program is read and controlled in its execution by the processor 1218. Intermediate storage of program data may be accomplished using RAM 1220. The method(s) of the example embodiments can be implemented as computer readable instructions, computer executable components, or software modules. One or more software modules may alternatively be used. These can include an executable program, a data link library, a configuration file, a database, a graphical image, a binary data file, a text data file, an object file, a source code file, or the like. When one or more computer processors execute one or more of the software modules, the software modules interact to cause one or more computer systems to perform according to the teachings herein.

The operation of the computer unit 1202 can be controlled by a variety of different program modules. Examples of program modules are routines, programs, objects, components, data structures, libraries, etc. that perform particular tasks or implement particular abstract data types. The example embodiments may also be practiced with other computer system configurations, including handheld devices, multiprocessor systems, microprocessor-based or programmable consumer electronics, network PCs, minicomputers, mainframe computers, personal digital assistants, mobile telephones and the like. Furthermore, the example embodiments may also be practiced in distributed computing environments where tasks are performed by remote processing devices that are linked through a wireless or wired communications network. In a distributed computing environment, program modules may be located in both local and remote memory storage devices.

### Applications

In the described example embodiments, the system and method utilize a combination of time-domain spectroscopy (e.g., terahertz time-domain spectrum measurement) and ultrasonic pressure loading to measure one or more mechanical properties (e.g., elasticity and rigidity) of a material (e.g., hydrogels).

In the described example embodiments, the system for non-contact measurement of elastic properties of a hydrogel mixture comprises (1) an ultrasonic system configured to generate ultrasonic pressure in the hydrogel mixture; (2) an illumination system configured to provide an illumination beam of terahertz electromagnetic radiation; (3) an optical system configured to form an optical path; (4) a terahertz detector configured to receive THz detection signals; (5) electronics configured to drive the ultrasonic system and to control a THz emitter and receiver; (6) a signal processing system configured to communicate with said terahertz detector to receive said THz detection signal, wherein said signal processing system processes said THz detection signal to provide a measure of elastic and rigidity in the hydrogel mixture.

Advantageously, embodiments of the system and method disclosed herein may provide a rapid and non-contact technique for measuring one or more mechanical properties of a material. In addition, the non-contact system and method may be used to monitor time-dependent changes of the material's properties.

Even more advantageously, embodiments of the system and method disclosed herein may provide a non-invasive and non-destructive technique for measuring one or more mechanical properties of a material, thereby rendering the system and method suitable for in-situ/in-vivo measurements, e.g., measurement of ambient tissue.

Accordingly, embodiments of the system and method disclosed herein may be employed in applications such as measurement of mechanical properties of a material, measurement of biosamples with high water content, evaluation of hydration level of a material, tissue hardness test, monitoring and measurement of tissue e.g., cornea properties.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A system for non-contact measurement of one or more mechanical properties of a material, the system comprising
an ultrasonic module comprising an ultrasonic applicator configured to apply ultrasonic pressure on a target region of the material;
a detection module comprising an electromagnetic wave emitter and an electromagnetic wave detector, said electromagnetic wave emitter being configured to emit an incident beam of electromagnetic waves towards the target region of the material, and said electromagnetic wave detector being configured to detect an emergent beam of electromagnetic waves reflected from the target region and/or transmitted through the target region; and
a processing module configured to determine one or more measures corresponding to the one or more mechanical properties of the material, based on changes in the emergent beam of electromagnetic waves.

2. The system according to claim 1, wherein the electromagnetic wave emitter and the electromagnetic wave detector are positioned such that the electromagnetic wave detector is capable of detecting the emergent beam of electromagnetic waves reflected from the target region.

3. The system according to claim 1, wherein the electromagnetic wave emitter and the electromagnetic wave detector are positioned such that the electromagnetic wave detector is capable of detecting the emergent beam of electromagnetic waves transmitted through the target region.

4. The system according to claim 3, wherein the electromagnetic wave emitter is positioned to emit an incident beam of electromagnetic waves that is substantially perpendicular to the surface of the material at the target region.

5. The system according to claim 1, wherein the ultrasonic applicator comprises an air-coupled ultrasonic transducer.

6. The system according to claim 1, wherein the ultrasonic applicator comprises an array of transducer elements configured to generate directed ultrasonic waves to a 3D spatial location at the target region.

7. The system according to claim 1, wherein the ultrasonic applicator comprises a pulsed laser device configured to emit pulses of electromagnetic waves for applying the ultrasonic pressure at the target region on the surface of the material.

8. The system according to claim 1, wherein the electromagnetic wave emitter is configured to emit an incident beam of electromagnetic waves having a frequency falling in a range from 0.1 THz to 10 THz.

9. The system according to claim 1, wherein the processing module is configured to perform time-domain spectrum measurement based on changes in the emergent beam of electromagnetic waves detected by the electromagnetic wave detector.

10. The system according to claim 1, wherein the one or more measures corresponding to the one or more mechanical properties of the material comprises a measure of elasticity, rigidity, viscoelasticity and/or rheology.

11. A method of non-contact measurement of one or more mechanical properties of a material, the method comprising,
emitting an incident beam of electromagnetic waves from an electromagnetic wave emitter towards a target region of the material;
applying or varying an ultrasonic pressure on the target region of the material;
detecting an emergent beam of electromagnetic waves reflected from the target region and/or transmitted through the target region with an electromagnetic wave detector; and
determining one or more measures corresponding to the one or more mechanical properties with a processing module, based on changes in the emergent beam of electromagnetic waves.

12. The method according to claim 11, wherein the changes in the emergent beam of electromagnetic waves are determined with respect to a reference emergent beam detected at a different time point.

13. The method according to claim 11, wherein the ultrasonic pressure applied at the target region on the surface of the material is substantially constant without amplitude modulations.

14. The method according to claim 11, wherein the method comprises,
(i) emitting the incident beam of electromagnetic waves and detecting the emergent beam of electromagnetic waves at a first time point;
(ii) varying the ultrasonic pressure on the target region of the material;
(iii) emitting the incident beam of electromagnetic waves and detecting the emergent beam of electromagnetic waves at a second time point;
(iv) repeating steps (i) to (iii) one or more times; and
(v) determining the one or more measures corresponding to the one or more mechanical properties based on differences in emergent beams at at least two different time points.

15. The method according to claim 11, wherein the ultrasonic pressure is applied using an air-coupled ultrasonic transducer over air.

16. The method according to claim 11, wherein the ultrasonic pressure is applied using a pulsed laser device over air, by applying pulses of electromagnetic waves having a pulse duration in the order of nanoseconds, picoseconds, or femtoseconds, and optionally wherein the pulses of electromagnetic waves are diffused to cover the target region on the surface of the material.

17. The method according to claim 11, wherein the incident beam of electromagnetic waves emitted by the electromagnetic wave emitter has a frequency falling in a range from 0.1 THz to 10 THz.

18. The method according to claim 11, further comprising performing time-domain spectrum measurement based on the emergent beam of electromagnetic waves detected by the electromagnetic wave detector before and after application of the ultrasonic pressure.

19. The method according to claim 11, wherein the material comprises a hydrogel or a soft tissue in an eye of a mammalian subject.

20. A computer readable storage medium having stored thereon instructions for instructing a processing unit of a system to execute a method of non-contact measurement of one or more mechanical properties of a material, the method comprising,
emitting an incident beam of electromagnetic waves from an electromagnetic wave emitter towards a target region of the material;
applying or varying an ultrasonic pressure on the target region of the material;
detecting an emergent beam of electromagnetic waves reflected from the target region and/or transmitted through the target region with an electromagnetic wave detector; and
determining one or more measures corresponding to the one or more mechanical properties with a processing module, based on changes in the emergent beam of electromagnetic waves.
